(19) United States
(12) Patent Application Publication
(10) Pub. No.: US 2020/0239210 A1
(43) Pub. Date: Jul. 30, 2020

(54) Wax Article and Packaging Therefor

(71) Applicant: Michael R. Schramm, Perry, UT (US)

(72) Inventor: Michael R. Schramm, Perry, UT (US)

(21) Appl. No.: 16/846,285

(22) Filed: Apr. 11, 2020

(51) Int. Cl. B65D 75/32; B65B 5/04; A61L 9/012

(52) U.S. Cl. CPC B65D 75/326; A61L 9/012; B65B 5/04

(57) ABSTRACT

A unitary scented wax article sized and shaped such that when melted, the volume of molten wax produced therefrom substantially matches the critical volume of a spill resistant container adapted to receive the wax article therein. The wax article further preferably “fits” within the spill resistant container as if it were cast in place in the spill resistant container. The invention further includes a packaged wax article apparatus comprising a flexible clear vacuum-formed plastic “clamshell” package having the wax article removably contained therein.

### FIELD OF THE INVENTION

The present invention relates to wax articles, and more particularly to wax articles for use in spill resistant containers and packaging therefor.

### BACKGROUND OF THE INVENTION

Spill resistant containers, especially containers having funnels impending therein are known in the art. Examples of such containers are disclosed in U.S. Pat. Nos. 5,246,046, 5,495,876, 5,832,969, 5,908,057, 6,386,138, 7,942,109, 8,430,708, 9,314,708, 10,322,200, 20020129763, 2014/0190858, 2015/0246294, 2015/0246295, 2015/0314320, 2016/0023822, 2016/0346709 and D660,076 all to Schramm and are expressly incorporated herein by reference. More specifically, a container having funnel impending therein and adapted to spill resistantly contain wax and fluent scented compositions are know in the art. An example of such container is disclosed in U.S. Pat. No. 10,322,200 to Schramm and is expressly incorporated herein by reference. Further, various wax and wax containing devices are known in the art. Examples of such wax and wax containing devices are disclosed in U.S. Pat. No. 10,039,851, 2006/0018786, 2015/0209463 and 2016/0214778 and are expressly incorporated herein by reference.

However, adapting spill resistant containers to aroma emitting fluent materials, and more especially to scented wax articles, creates problems not solved by the prior art. For instance, unlike paint, liquid dye or bubble solution, wax is typically placed in a container in a solid state, and then is liquified (melted) thereafter. When wax melts, wax swells or increases in volume. Further, wax is commonly provided in so-called “ice cube trays” which typically comprise an article of wax formed such that several cubes joint by thin members can be readily broken off or separated from the remaining cubes (i.e. as if the wax were formed in an ice cube tray). These cubes are then typically added to a container with little concern for either the melted or unmelted volume thereof. It is noted that it is typically desirable to place a maximal amount of wax in a container so as to maximize the emitted aroma or “scent throw”. However, when using a spill resistant container having a funnel impending therein as the spill resistant container for wax, it is important to not exceed a “critical volume” of the container with a liquid contents of the container. It shall be noted that a “critical volume” of such container is equal to a maximum volume of liquid contents of the container wherein when the container is rotated or oriented in any position, the liquid will not run out of the container. It shall also be noted that in order to minimize the overall size of a spill resistant container, the container is often geometrically efficient—meaning that there is little extra or unused space within the container. Thus with prior art devices, not only is one left to guess as to how much solid wax—especially a plurality of separate wax members—to place in a spill resistant wax container so as not to exceed the critical volume of the container, because of the efficiency of the design of the container, one may not be able to place enough solid wax in the container to maximum the aroma emission or scent throw of the container apparatus.

### SUMMARY OF THE INVENTION

The present invention is a wax article expressly adapted such that when the wax article is in a liquified (melted) state, the volume of the liquified wax thereof substantially matches a predetermined critical volume of a spill resistant container in which it is intended that the wax article will be used. To achieve the aforementioned objective, the wax article is preferably adapted such that all distinct surfaces but one of the wax article substantially match to a surface of the spill resistant container in which it is intended that the wax article will be used, much as if the wax article were “cast” or poured into the container in a liquified state and then allowed to solidify. The invention further preferably includes packaging for the wax article which in a preferred embodiment comprises a plastic vacuum-formed clamshell adapted to preferably provide a “see through” visual display of the wax article and so as to allow for hanging on a commercial display rack.

### DESCRIPTION OF DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a trimetric view of a spill resistant candle apparatus of a first embodiment of the invention having the wax article container therein in an assembled configuration;

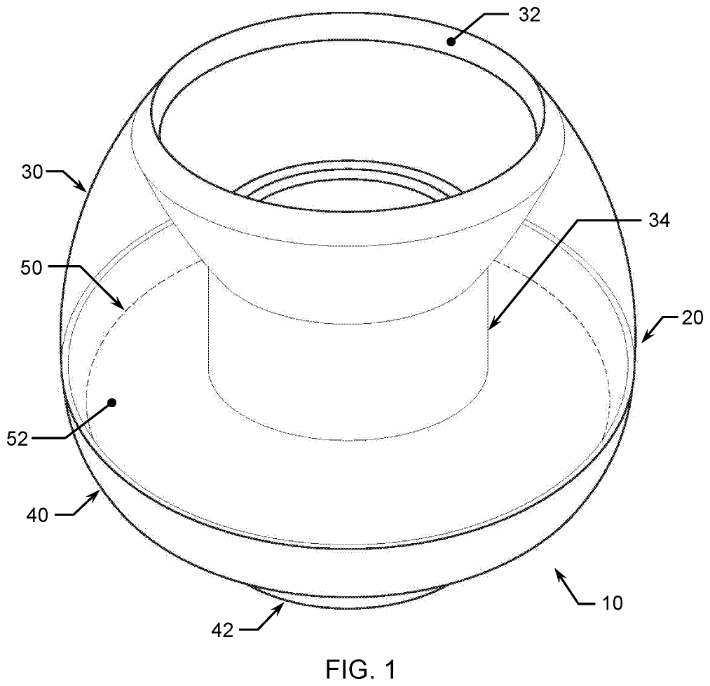

FIG. 2 is an exploded trimetric view of the spill resistant candle apparatus of the first embodiment of the invention showing the article positioned between an upper portion of the container and a lower portion of the container;

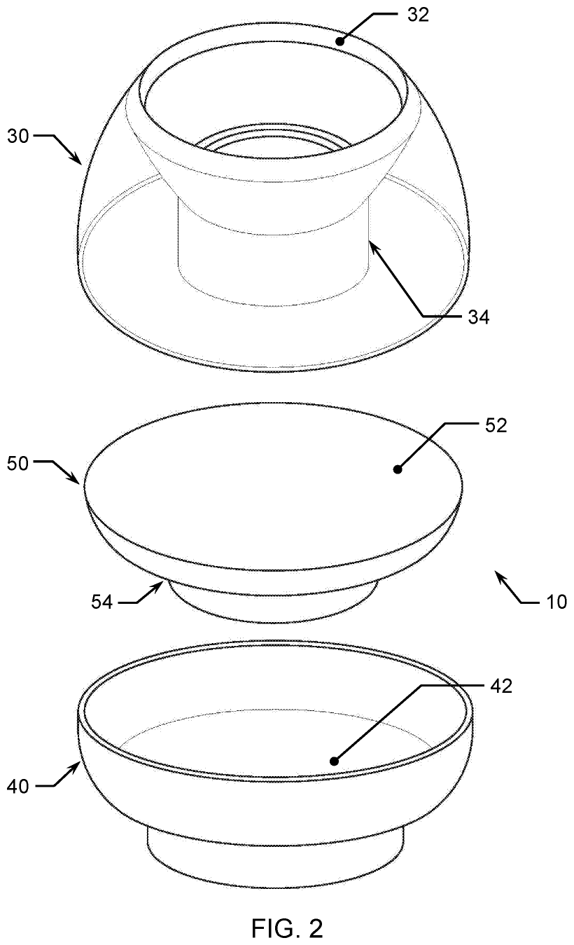

FIG. 3 is a first trimetric view of a packaged wax article apparatus of the first embodiment of the invention having the wax article contained therein and showing a front side of the packaged wax article apparatus;

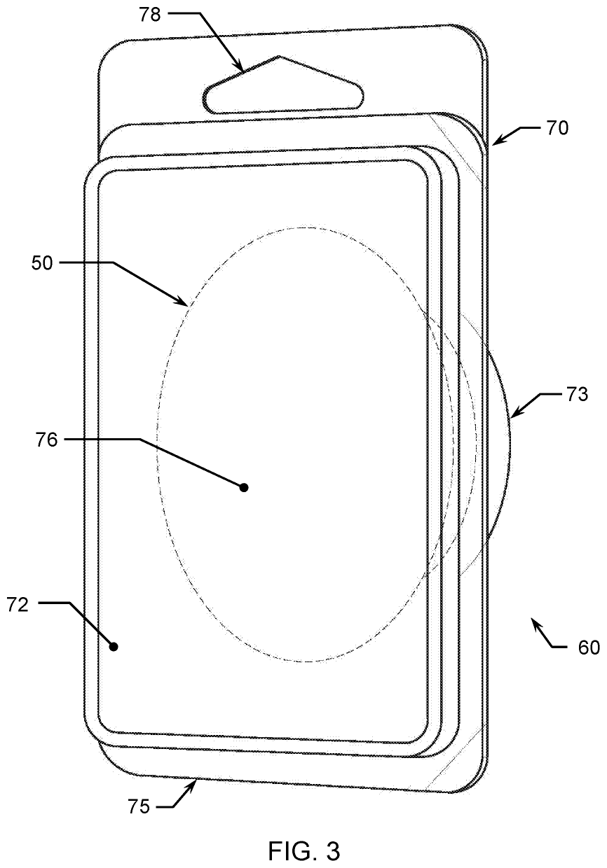

FIG. 4 is a second trimetric view of a packaged wax article apparatus of the first embodiment of the invention having the wax article contained therein and showing a back side of the packaged wax article apparatus, and;

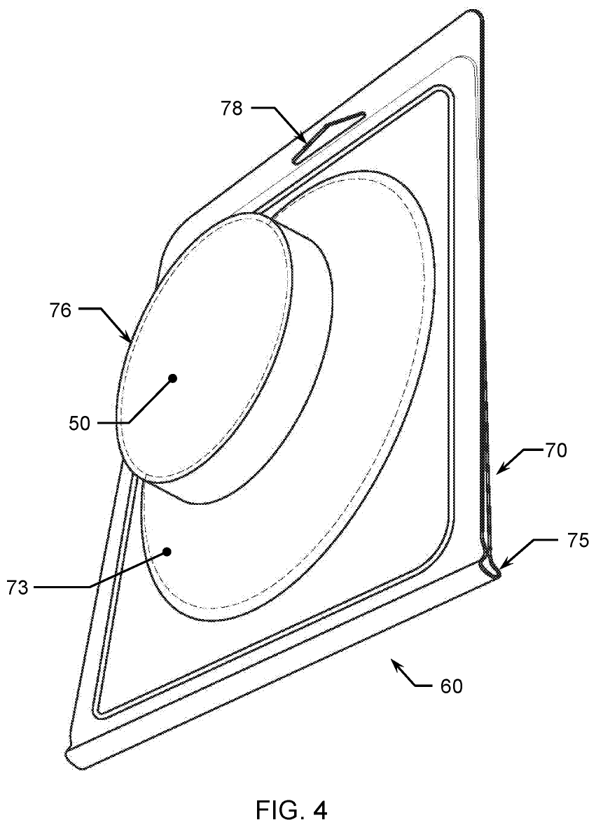

FIG. 5 is a trimetric view of an alternate wax article having a wick.

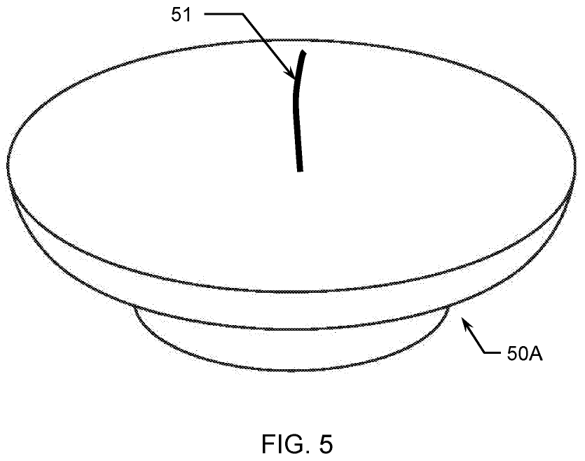

### DETAILED DESCRIPTION OF THE INVENTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are included to provide a thorough understanding of embodiments of the invention. One skilled in the relevant art will recognize, however, that the invention can be practiced without one or more of the specific details, or with other methods, components, materials, and so forth. In other instances, well-known structures, materials, or operations are not shown or described in detail to avoid obscuring aspects of the invention.

In order to facilitate the understanding of the present invention in reviewing the drawings accompanying the specification, a feature table is provided below. It is noted that like features are like numbered throughout all of the figures.

FEATURE TABLE

| # | Feature | # | Feature |
|---|---|---|---|
| 10 | Spill resistant candle | 20 | Spill resistant container |
| | apparatus | | |
| 30 | Container upper member | 32 | Upper member upper opening |
| 34 | Upper member funnel | 40 | Container lower member |
| 42 | Lower member well | 50 | Wax article |
| 50A | Wax article | 51 | Wick |
| 52 | Wax article upper surface | 54 | Wax article lower surface |
| 60 | Packaged wax article | 70 | Package |
| | apparatus | | |
| 72 | Package front portion | 73 | Package back portion |
| 75 | Package hinge | 76 | Package pocket |
| 78 | Package cutout | | |

Referring now to the drawings, in a preferred embodiment the invention is a wax article 50 for use in a spill resistant container apparatus 10. Spill resistant container apparatus 10 preferably comprises a resistant container 20 having wax article 50 contained therein. Spill resistant container 20 preferably comprises a container upper member 30 and a container lower member 40 and is adapted such that upper member 30 and container lower member 40 are removably assembled together via threads to provide a threaded connection. When spill resistant container 20 is assembled, spill resistant container 20 has a “critical volume”. Upper member 30 further includes an upper opening 32 and a funnel 34 connected to upper opening 32. Lower member 40 further includes a well 42 formed therein. Wax article 50 preferably comprises a meltable unitary scented wax article having an upper surface 52 and a lower surface 54. Wax article 50 is preferably of a size and shape such that when melted, the volume of molten wax produced thereby is substantially equal to the “critical volume” of resistant container 20. Wax article 50 is preferably generally disc shaped. It shall be noted however, that the volume of molten wax of wax article 50 may be somewhat less than or more than the “critical volume”. For instance the volume of molten wax of wax article 50 may be for instance 1%, 2%, 3%, 5%, 8%, 10%, 12%, 15%, 20%, 25% and 50% or more greater or less than the “critical volume”. Further the volume of molten wax produced from melting wax article 50 may be for instance in the range of 0.5-5 ounces, 1-4 ounces, 1.5-3.5 ounces, 1.75-3 ounces, 2-2.75 ounces, and 2.2-2.7 ounces, or volume greater or less than such ranges. Wax article 50 is further preferably of a size and shape such that upper surface 52 is substantially flat and lower surface 54 substantially conforms to lower member well 42 or when placed in lower member 40, substantially all of lower surface 54 is substantially in faying contact with lower member well 42. Described differently, wax article 50 is of a size and shaped as if it were cast in place in lower member well 42. Wax article 50 preferably free of (does not include any) connecting members such as those thin connecting members common to so-called “ice-cube” trays of wax and which connect individual cubes of wax in a an “ice-cube” tray of wax and which function as convenient breakpoints to facilitate breaking the “ice-cube” tray of wax into individually separated and discrete cubes or portions of wax. As an alternative to wax article 50, wax article 50A may be provided and used in place of wax article 50. Wax article 50A is substantially identical to wax article 50 except that wax article 50A includes an ignitable wick 51 protruding therefrom.

Further referring to the drawings, in a preferred embodiment the invention includes a packaged wax article apparatus 60 comprising package 70 containing wax article 50 therein. Package 70 preferably comprises a flexible clear vacuum-formed plastic “clamshell” package having a front portion 72, a back portion 73, a (live) hinge 75, a pocket 76 and a cutout 78. Pocket 74 is sized and shaped to receive wax article 50 therein, hinge 50 is adapted to flex, front portion 72 and back portion 73 include sealing edges thereon, such that and package 70 substantially seals wax article 50 therein by snappingly engaging front portion 72 to back portion 73 and such that wax article 50 removable therefrom by unsnapping front portion 72 from back portion 73. Cutout 78 is formed in an upper portion of back portion 73 and adapted such that packaged wax article apparatus 60 may be displayingly hung from a wire rack such as are common in mass market retail stores.

In practice, when the scent emitted from spill resistant container apparatus 10 reduces to the point that a user desires to “service” spill resistant container apparatus 10, a services spill resistant container apparatus 10 as follows. Upper member 30 is unthreaded from lower member 40 and separated from lower member 40. Spent wax article 50 is removed from lower member 40. If required for easy extraction, lower member 40 with spent wax article 50 therein is placed in a freezer for a period of time (e.g. an hour) and then lower member 40 is removed from a freezer and tapped on a hard surface or object such that spent wax article 50 pops out of lower member 40. Spent wax article 50 is discarded. A packaged wax article apparatus 60 having a fresh wax article 50 contained therein is opened by unsnapping front portion 72 from back portion 73 and removing fresh wax article 50 therefrom and placing fresh article 50 in (now empty) lower member 40. At this point spill resistant container apparatus 10 is serviced and ready to be placed on a warmer to cause additional scent throw. It shall be note that wax article 50 allows a user to service spill resistant container apparatus 10 without measuring wax and without guessing as to how much wax to place in spill resistant container 20 and without the concern for overfilling spill resistant container 20 which could result in spilled molten wax and without concern for under filling spill resistant container 20 which could result in insufficient scent throw and excessively frequent servicing of spill resistant container apparatus 10.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A wax article comprising at least one of:
a wax article having a shape wherein all but one surface of said wax article substantially fits to and matches the shape of a lower portion of a spill resistant container adapted to receive said wax article,
a wax article adapted such that when melted, the volume of molten wax produced therefrom substantially matches a critical volume of a spill resistant container adapted to receive said wax article,
and a unitary wax article adapted to fit within a lower portion of a spill resistant container adapted to receive said unitary wax article, wherein unitary wax article is further adapted such that when melted, the volume of molten wax produced therefrom comprises at least one of a volume of 0.5-5 ounces, a volume of 1-4 ounces, a volume of 1.5-3.5 ounces, a volume of 1.75-3 ounces, a volume of 2-2.75 ounces, and a volume of 2.2-2.7 ounces.

2. The wax article of claim 1, wherein when heated, said wax article emits a predetermined scent.

3. The wax article of claim 1, wherein said wax article is unitarily packaged.

4. The wax article of claim 3, wherein said package comprises a clamshell package.

5. The wax article of claim 3, wherein said package is at least partially transparent so as to at least partially reveal said wax article contained therein.

6. The wax article of claim 3, wherein said wax article comprises at least one of a wickless wax article and a wax article having a wick.

7. The wax article of claim 1, wherein a range of said volume of said molten wax comprises 2.2 ounces to 2.7 ounces, and wherein said wax article is unitarily packaged, and wherein said package comprises an at least partially transparent clamshell package adapted so as to at least partially reveal said wax article contained therein, and wherein said wax article comprises a wickless wax article that emits a predetermined scent when heated.

8. Packaged wax, wherein said wax comprises at least one of:
a wax article having a shape wherein all but one surface of said wax article substantially fits to and matches the shape of a lower portion of a spill resistant container adapted to receive said wax article,
a wax article adapted such that when melted, the volume of molten wax produced therefrom substantially matches a critical volume of a spill resistant container adapted to receive said wax article,
and a unitary wax article adapted to fit within a lower portion of a spill resistant container adapted to receive said unitary wax article, wherein unitary wax article is further adapted such that when melted, the volume of molten wax produced therefrom comprises at least one of a volume of 0.5-5 ounces, a volume of 1-4 ounces, a volume of 1.5-3.5 ounces, a volume of 1.75-3 ounces, a volume of 2-2.75 ounces, and a volume of 2.2-2.7 ounces.

9. The wax article of claim 8, wherein when heated, said wax article emits a predetermined scent.

10. The packaged wax of claim 8, wherein said wax article is unitarily packaged.

11. The packaged wax of claim 8, wherein said package comprises a clamshell package.

12. The packaged wax of claim 8, wherein said package is at least partially transparent so as to at least partially reveal said wax article contained therein.

13. The packaged wax of claim 8, wherein said wax article comprises at least one of a wickless wax article and a wax article having a wick.

14. The packaged wax of claim 8, wherein a range of said volume of said molten wax comprises 2.2 ounces to 2.7 ounces, and wherein said wax article is unitarily packaged, and wherein said package comprises an at least partially transparent clamshell package adapted so as to at least partially reveal said wax article contained therein, and wherein said wax article comprises a wickless wax article that emits a predetermined scent when heated.

15. A method of servicing a spill resistant container comprising the steps of providing a spill resistant container and at least one of:
a wax article having a shape wherein all but one surface of said wax article substantially fits to and matches the shape of a lower portion of a spill resistant container adapted to receive said wax article,
a wax article adapted such that when melted, the volume of molten wax produced therefrom substantially matches a critical volume of a spill resistant container adapted to receive said wax article,
and a unitary wax article adapted to fit within a lower portion of a spill resistant container adapted to receive said unitary wax article, wherein unitary wax article is further adapted such that when melted, the volume of molten wax produced therefrom comprises at least one of a volume of 0.5-5 ounces, a volume of 1-4 ounces, a volume of 1.5-3.5 ounces, a volume of 1.75-3 ounces, a volume of 2-2.75 ounces, and a volume of 2.2-2.7 ounces, and;
placing said article of wax in said spill resistant container.

16. The method of claim 15, wherein said step of placing is preceded by a step of removing used wax from said spill resistant container.

17. The method of claim 15, wherein said wax article is unitarily packaged and said method includes the step of removing said wax article from said packaging.

18. The method of claim 15, wherein when heated, said wax article emits a predetermined scent.

19. The method of claim 17, wherein said package comprises a clamshell package.

20. The method of claim 15, wherein a range of said volume of said molten wax comprises 2.2 ounces to 2.7 ounces, and wherein said wax article is unitarily packaged, and wherein said package comprises an at least partially transparent clamshell package adapted so as to at least partially reveal said wax article contained therein, and wherein said wax article comprises a wickless wax article that emits a predetermined scent when heated.
